(19) United States
(12) Patent Application Publication
(10) Pub. No.: US 2003/0077312 A1
(43) Pub. Date: Apr. 24, 2003

(54) Coated intraluminal stents and reduction of restenosis using same

(76) Inventors: Ascher Schmulewicz, Tel Aviv (IL); Gideon Strassman, Washington, DC (US)

Correspondence Address: SCHNADER HARRISON SEGAL & LEWIS, LLP, 1600 MARKET STREET, SUITE 3600, PHILADELPHIA, PA, 19103

(21) Appl. No.: 10/054,110

(22) Filed: Oct. 22, 2001

(51) Int. Cl. A61M 29/00; A61K 31/7076; A61K 31/4015

(52) U.S. Cl. 424/426; 604/103.06; 424/756; 514/46; 514/424; 514/559

(57) ABSTRACT

Medical devices such as intracoronary stents coated with a therapeutic substance are disclosed. In a preferred embodiment, an arterial site with obstructive coronary artery disease is treated via a therapeutic substance applied to an intraluminal stent and placed locally in the coronary artery. Improved porous designs polymeric films and coatings for stents, with or without a therapeutic substance, are also disclosed. By providing a separate sleeve and stent, various bioactive materials can be impregnated into the sleeve and combined with the stent, providing greater variety to treatment options, and significant improvements in regulatory protocols as new bioactive or therapeutic materials are produced. The present invention provides a stent that has a substrate and a degradable sleeve, and the sleeve itself is made of a carrier material, such as the polymeric materials and a bioactive compound. Certain bioactive materials have been found to be useful for impregnation into stent coatings or sleeves, such as rolipram, phosphodiesterase type IV inhibitors, curcumin, adenosine and adenosine receptor type 2A agonists, all of which have now been found to significantly reduces restenosis. Alternatively, the present invention, in another aspect, also relates to the promotion of angiogenesis on stents, by inserting a stent into a vessel where the stent has a substrate and a coating, wherein the coating is selected from the group: retinoic acid, Matrigel, laminin and laminin derived peptides.

### BACKGROUND OF THE INVENTION

1. Field of the Invention

The present invention relates medical devices and more particularly to medical devices coated with a therapeutic substance. In a preferred embodiment, an arterial site with obstructive coronary artery disease is treated via a therapeutic substance applied to an intraluminal stent and placed locally in the coronary artery. Improved porous designs polymeric films and coatings for stents, with or without a therapeutic substance, are also disclosed.

2. Brief Description of the Prior Art

The narrowing or constriction of a vessel is typically treated via percutaneous transluminal coronary angioplasty (PTCA) with the insertion and inflation of a balloon catheter into a stenotic vessel. However, restenosis at the site of a prior invasive coronary artery disease therapy can occur. Restenosis is the recurrence of a 50% or greater narrowing of a luminal diameter at the site of a prior balloon dilatation. Angioplasty or other vascular surgeries injure the arterial wall, removing the vascular endothelium, disturbing the underlying intima and causing death of medial smooth muscle cells. Excessive neointimal tissue formation, characterized by smooth muscle cell migration and proliferation into the intima, follows the injury. The extensive thickening of this tissue narrows the lumen of the blood vessel, constricting or blocking blood flow through the artery. This phenomenon is sometimes referred to as “intimal hyperplasia.” It is believed that a variety of biologic factors are involved in restenosis, such as the extent of the injury, platelets, inflammatory cells, growth factors, cytokines, endothelial cells, smooth muscle cells, and extracellular matrix production.

Attempts to inhibit or diminish restenosis often include additional interventions such as the use of intravascular stents applied over a PTCA balloon and radially expanded, such as those disclosed in U.S. Pat. No. 4,733,665—Palmaz and U.S. Pat. No. 4,800,882—Gianturco. Stents are “scaffoldings,” usually cylindrical or tubular in shape, which function to physically hold open or even expand the lumen of a vessel. Typically stents are compressible, so that they can be inserted through small cavities via small catheters, and then expanded to a larger diameter once they are delivered to a desired location. Stents are also capable of carrying therapeutic substances and locally releasing such substances for a predetermined duration of time. This allows high concentrations of therapeutic substances to be delivered directly to a treatment site. Stents employing therapeutic substances such as glucocorticoids (e.g. dexamethasone, beclamethasone), heparin, hirudin, tocopherol, angiopeptin, aspirin, ACE inhibitors, growth factors, oligonucleotides, and, more generally, antiplatelet agents, anticoagulant agents, antimitotic agents, antioxidants, antimetabolite agents, and anti-inflammatory agents have been considered for their potential to solve the problem of restenosis. Typically, such substances have been incorporated into a solid composite with a polymer in an adherent layer on a stent body with fibrin in a separate adherent layer on the composite to form a two layer system. The fibrin is optionally incorporated into a porous polymer layer in this two layer system. One example of a coated stent is U.S. Pat. 5,900,246—Lambert which discloses biologically active compounds such as lipophilic compounds, for example, Forskolin, sphingosine, etretinate, lipid modified and oligonucleotides.

Another concern with intravascular and extracorporeal procedures is the contact of biomaterials with blood, which can trigger the body's hemostatic process. The hemostatic process is normally initiated as the body's response to injury. When a vessel wall is injured, platelets adhere to damage endothelium or exposed subendothelium. Following adhesion of the platelets, these cells cohere to each other preparatory to aggregation and secretion of their intracellular contents. Simultaneously there is activation, probably by electrostatic charge of the contact factors, of the coagulation cascade. The sequential step-wise interaction of these procoagulant proteins results in the transformation of soluble glycoproteins into insoluble polymers, which after transamidation results in the irreversible solid thrombus.

When restenosis does occur in the stented segment, its treatment can be challenging, as clinical options are more limited as compared to lesions that were treated solely with a balloon. A method for inhibiting restenosis at a stent implantation site would reduce the mortality rate associated with restenosis. To inhibit restenosis, therapeutic agents hoped to counter important steps in the formation of the neointimal tissue are being developed, particularly those that inhibit the migration and proliferation of smooth muscle cells. For example, platelet derived growth factor (PDGF) stimulates smooth muscle cell growth at arterial lesions; the administration of monoclonal anti-PDGF receptor antibodies is being advanced. Similarly, secretory T lymphocyte protein interferon-gamma, which has also been shown to inhibit smooth muscle growth, is being tested, but so far is unable to adequately inhibit restenosis. Additional pharmacological therapies, such as the administration of heparin to inhibit thrombus formation, calcium channel blockers to reduce platelet aggregation, and angiotensin agonists to prevent vasoconstriction have also met with limited success.

Therefore, there is a need to sufficiently inhibit restenosis at a stent site, to greatly improve the effectiveness of coronary stents, and to improve the effectiveness of any long-term or permanent devices implanted within a blood vessel. There is also a need for a better active composition to inhibit restenosis.

It has been suggested that retinoids inhibit early stage angiogenesis, mainly via vascular endothelial growth factor (VEGF) inhibition; however, these compounds also promote fibrin growth (via FGF-2), in the context of an intracoronary stent. It is thought that the more relevant effect is that retinoids inhibit smooth muscle proliferation. U.S. Pat. No. 6,261,585—Sefton et al. discloses using retinoic acid as an anti-angiogenic factor, and tumor growth inhibitor. This reference groups retinoic acid with Anti-Invasive Factor, paclitaxel, Suramin, Tissue Inhibitor of Metalloproteinase-1, Tissue Inhibitor of Metalloproteinase-2, Plasminogen Activator Inhibitor-1 and Plasminogen Activator Inhibitor-2, and lighter “d group” transition metals. However, the effect of retinoic acid (RA) on endothelial cells is controversial and it is thought that retinoic acid can stimulate endothelial cell proliferation and differentiation in vitro via enhanced RARalpha-dependent FGF-2 production, and it can also induce angiogenesis in vivo. Gaetano, et al., Circ. Res. 88: 38e-47e (2001) “Retinoids induce fibroblast growth factor-2 production in endothelial cells via retinoic acid receptor alpha activation and stimulate angiogenesis in vitro and in vivo.” The full text of this article is available at http://www.circresaha.org. It is also known that retinoids exert anti-proliferative and pro-differentiating effects in vascular smooth muscle cells and reduce neointimal mass in

Alternatively, the present invention, in another aspect, also relates to the promotion of angiogenesis on stents, by inserting a stent into a vessel where the stent has a substrate and a coating, wherein the coating is selected from the group: retinoic acid, Matrigel, laminin and laminin derived peptides.

### BRIEF DESCRIPTION OF THE DRAWINGS

FIG. 1 is a perspective view of an un-expanded stent made in accordance with the present invention;

FIG. 2 is a perspective view of a sleeve for the stent shown in FIG. 1;

FIG. 3 is a cross-sectional view of the stent and sleeve shown in FIGS. 1-2 when expanded within a vessel of a patient; and

FIG. 4 is a detailed section taken at 4-4 of FIG. 3 illustrating the migration of therapeutic substances into the vessel wall.

### DETAILED DESCRIPTION OF THE PREFERRED EMBODIMENTS

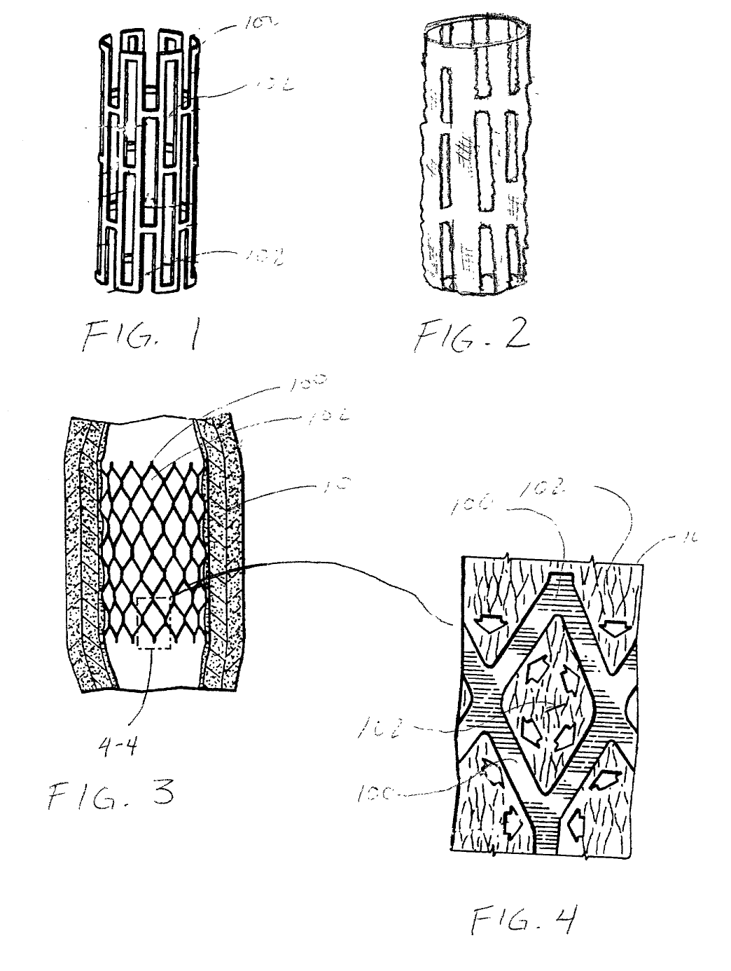

Referring now to FIG. 1 there is shown a perspective view of an un-expanded stent 100 made in accordance with the present invention. The stent 100 typically is comprised of stainless steel or other malleable material that is biocompatible and will expand to a larger diameter to enlarge the lumen of a body vessel. Openings 102 permit expansion to a wire frame shape, discussed below with reference to FIG. 3. The function and construction of such stents is well known in the art and stents of varying types, sizes and designs, for varying indications are widely available. The stent 100 shown in FIG. Is for purposes of illustration and is not meant to be limiting. As explained below, it will be desirable to combine the basic therapy of a stent with a therapeutic substance. Such combination can be a coating that is not visible, and hence not illustrated in FIG. 1

Alternatively and in accordance with one aspect of the present invention, a sleeve containing a therapeutic substance can be added, as seen FIG. 2 which is a perspective view of a sleeve 200 for the stent 100 shown in FIG. 1. The sleeve 200 may be made of any suitable material that is biocompatible and will bind with the therapeutic material, or in some cases can be made from biocompatible material itself. The sleeve will preferably be elastic or malleable so that it can conform to the stent both before and after expansion without cracking, breaking, pulverizing or otherwise degenerating. Typically, the sleeve will be formed from a polymeric material. Polymeric and other materials suitable for the sleeve 200 are well known in the art, and are used for a variety of purposes, including grafts and other implant able devices.

As will be readily understood by those skilled in the art, the design of the openings 102 in the stent 100 and the sleeve may be arrayed as seen fit by the designers. Certain embodiments of the sleeve 102 will be a mesh, woven or non-woven, while others will be sintered, molded, or formed such that the sleeve ahs fenestrations that register with the openings 102 of the stent. In this latter regard, reference is made to FIGS. 3-4. In FIG. 3, a vessel 10 is illustrated in cross-section with the stent shown in FIG. 1 in an expanded state. As seen in the detail of FIG. 4, the stent 100 overlies part of the vessel wall and the openings 102 thus present an opportunity to either permit the vessel wall to directly contact blood, or, alternatively, can be covered by a sleeve that is either not fenestrated or has fenestrations that do not register with the openings 102. The arrows in FIG. 4 illustrate the phenomenon explained below whereby therapeutic substances are absorbed and administered by the placement of either a coated stent or a stent with a sleeve.

Those of skill in the art will appreciate that numerous designs of stent scaffoldings are known, as well as numerous materials suitable for making such scaffoldings. Moreover, as noted above, numerous suitable coatings or sleeves can be adhered to, applied to, formed on or delivered with a stent. The present invention, as described below, is useful with any number of combinations of scaffoldings and coatings; for example, a typical embodiment is a polyurethane-coated Nitinol stent.

Additionally, it will be appreciated that it is important to quantify tissue uptake of any bioactive substance delivered via a stent. The determination of concentrations of a drug or other substance delivered to a vessel wall is readily determined using well know techniques and does not require undue experimentation. In particular, it is useful to determine radial and longitudinal diffusion at various points proximal, distal, and radial to the stent show that there is a diffusion gradient in both longitudinal and radial directions away from the stent to demonstrate that a coated stent is capable of delivering a drug in high local concentration in the vessel wall.

In order to provide the coated stent according to the present invention, a solution that includes a solvent, a polymer dissolved in the solvent and a therapeutic substance dispersed in the solvent is prepared. It is important to choose a solvent, a polymer and a therapeutic substance that are mutually compatible. It is essential that the solvent is capable of placing the polymer into solution at the concentration desired in the solution. It is also essential that the solvent and polymer chosen do not chemically alter the therapeutic character of the therapeutic substance. However, the therapeutic substance only needs to be dispersed throughout the solvent so that it may be either in a true solution with the solvent or dispersed in fine particles in the solvent.

The solution is applied to the stent and the solvent is allowed to evaporate, thereby leaving on the stent surface a coating of the polymer and the therapeutic substance. Typically, the solution can be applied to the stent by either spraying the solution onto the stent or immersing the stent in the solution. Whether one chooses application by immersion or application by spraying depends principally on the viscosity and surface tension of the solution, however, it has been found that spraying in a fine spray such as that available from an airbrush will provide a coating with the greatest uniformity and will provide the greatest control over the amount of coating material to be applied to the stent. In either a coating applied by spraying or by immersion, multiple application steps are generally desirable to provide improved coating uniformity and improved control over the amount of therapeutic substance to be applied to the stent.

The polymer chosen must be a polymer that is biocompatible and minimizes irritation to the vessel wall when the stent is implanted. The polymer may be either a biostable or a bioabsorbable polymer depending on the desired rate of release or the desired degree of polymer stability, but a bioabsorbable polymer is probably more desirable since, unlike a biostable polymer, it will not be present long after implantation to cause any adverse, chronic local response. Bioabsorbable polymers that could be used include poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid. Also, biostable polymers with a relatively low chronic tissue response such as polyurethanes, silicones, and polyesters could be used and other polymers could also be used if they can be dissolved and cured or polymerized on the stent such as polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

The ratio of therapeutic substance to polymer in the solution will depend on the efficacy of the polymer in securing the therapeutic substance onto the stent and the rate at which the coating is to release the therapeutic substance to the tissue of the blood vessel. More polymer may be needed if it has relatively poor efficacy in retaining the therapeutic substance on the stent and more polymer may be needed in order to provide an elution matrix that limits the elution of a very soluble therapeutic substance. A wide ratio of therapeutic substance to polymer could therefore be appropriate and could range from about 10:1 to about 1:100.

In accordance with one aspect of the present invention, in addition to the coatings discussed above, a separate sleeve made of a sheet of similar materials impregnated with similar bioactive or therapeutic substance can be formed and crimped or otherwise affixed to the substrate or scaffolding of the stent itself. It will be appreciated that this technique, as opposed to in situ formation or deposition of a coating, allows various compounds to be formulated and placed on a stent independent of the stent design. This will permit greater flexibility in stent design, construction and manufacture and will also permit unique regulatory protocols whereby a stent/sleeve combination may be approved, and then used with approved classes of drugs, therapeutics, bioactive materials, etc. Thus, a manufacturer may advantageously change the material impregnated in the sleeve and combine the sleeve with a stent without incurring the costs and delay heretofore required to gain regulatory approval. This aspect becomes increasingly important as the physicians who insert stents become accustomed to the mechanical aspect of a certain design, and wish to continue to use that design with an ever-changing and continually widening array of materials impregnated into the sleeve carried by the stent.

Whether formed upon the substrate or scaffolding of stent material itself, or attached by a crimped sleeve of impregnated material, it is preferred that the sleeve or polymer containing the bioactive or therapeutic substance degrade, or biodegrade over time to both assist in the release of the impregnated substance and to improve the long term stability and compatibility of the stent.

Thus, in a preferred embodiment, the present invention provides a stent that has a substrate and a degradable sleeve, and the sleeve itself is made of a carrier material, such as the polymeric materials discussed above and a bioactive compound. In certain embodiments, it will be desirable to use a sleeve that is pre-formed with a plurality of fenestrations, and in certain of these embodiments, it will further be desirable to have the fenestrations disposed adjacent openings in the stent, so that when the stent is expanded the fenestrations and openings are in registration. On the other hand, it will also be a useful embodiment to provide a sleeve with fenestrations disposed adjacent solid portions of the stent, so that upon expansion the fenestrations and solid portions are substantially in registration. Typically, it will be preferred to provide a sleeve that has a thickness of about 20-100 microns and that the sleeve is crimped to a delivery system and to said stent, although other thick nesses and methods of affixation can be used.

In accordance with another aspect of the present invention, certain materials have been found to be useful for impregnation into stent coatings or sleeves, as discussed above. In preferred embodiments of this aspect of the present invention, there are provided methods for inhibiting stent-related inflammation by inserting a stent into a vessel, where the stent has a substrate and a coating selected from the group: rolipram, phosphodiesterase type IV inhibitors, curcumin, adenosine and adenosine receptor type 2A agonists, all of which have now been found to significantly reduces restenosis.

Alternatively, the present invention, in another aspect, also relates to the promotion of angiogenesis on stents, by inserting a stent into a vessel where the stent has a substrate and a coating, wherein the coating is selected from the group: retinoic acid, Matrigel, laminin and laminin derived peptides.

Although certain embodiments of the present invention have been set forth herein with particularity, it will be appreciated from the foregoing descriptions of the preferred embodiments that numerous modifications, adaptations and substitutions readily present themselves to those of skill in the art which do no not depart from the spirit of the invention disclosed herein. Therefore, in order to ascertain the true scope of the present invention, reference should be made to the appended claims.

What is claimed is:

1. A stent comprising a substrate and a degradable sleeve, said sleeve comprising a carrier material and a bioactive compound.

2. The stent of claim 1 wherein the sleeve is pre-formed with a plurality of fenestrations.

3. The stent of claim 2 wherein said fenestrations are disposed adjacent openings in said stent, whereby upon expansion, said fenestrations and said opening are substantially in registration.

4. The stent of claim 2 wherein said fenestrations are disposed adjacent solid portions of said stent, whereby upon expansion, said fenestrations and said solid portions are substantially in registration.

5. The stent of claim 1 wherein the sleeve has a thickness of about 20-100 microns.

6. The stent of claim 1 wherein the sleeve is crimped to a delivery system and to said stent.

7. The stent of claim 6 wherein said delivery system is a balloon catheter.

8. The stent of claim 1 wherein the bioactive compound is selected from the group consisting of rolipram, phosphodiesterase type IV inhibitors, curcumin, adenosine and adenosine receptor type 2A agonists.

9. The stent of claim 1 wherein the bioactive compound is selected from the group consisting of retinoic acid, Matrigel, laminin and laminin derived peptides.

10. The stent of claim 1 wherein said substrate is comprised of metal.

11. A drug delivery system for localized delivery of a biologically active compound to a subject, comprising:
a substrate and a polymeric coating and at least one biologically active compound absorbed into the interstices of said coating.

12. The drug delivery system of claim 11 wherein the biological agent is absorbed substantially throughout the entire thickness of the coating.

13. The drug delivery system of claim 12 wherein the polymer coating has a thickness in the range of about 20 up to 100 microns.

14. The drug delivery system of claim 11, wherein the polymeric coating is crimped to said substrate.

15. The drug delivery system of claim 11, wherein the polymeric coating is formed on said substrate.

16. The drug delivery system of claim 11, wherein said biological agent is selected from the group consisting of rolipram, phosphodiesterase type IV inhibitors, curcumin, adenosine and adenosine receptor type 2A agonists.

17. The drug delivery system of claim 11, wherein said biological agent is selected from the group consisting of retinoic acid, Matrigel, laminin and laminin derived peptides.

18. A method for inhibiting stent-related inflammation, comprising the step inserting into a vessel a stent comprising a substrate and a coating selected from the group: rolipram, phosphodiesterase type IV inhibitors, curcumin, adenosine and adenosine receptor type 2A agonists.

19. The method of claim 18, wherein said vessel contains a lesion a least partially occluding the lumen of the vessel, and said stent increases the diameter of said lumen, whereby the coating significantly reduces restenosis.

20. A method for the promotion of angiogenesis on stents, comprising the step of inserting into a vessel a stent comprising a substrate and a coating, wherein the coating is selected from the group: retinoic acid, Matrigel, laminin and laminin derived peptides.

21. The method of claim 20, wherein said vessel contains a lesion a least partially occluding the lumen of the vessel, and said stent increases the diameter of said lumen, whereby the coating significantly reduces restenosis.

22. A method of increasing blood flow to a ischemic tissue comprising the step of implanting an angiogenic material into the ischemic animal tissue or blood vessels in the immediate vicinity of the ischemic animal tissue, said angiogenic material consisting of a biocompatible polymer and a vascularizing compound capable of promoting the growth of blood vessels, which, when implanted in said tissue, said angiogenic material promotes generation of blood vessels in its immediate vicinity and induces minimal or no fibrous capsule formation.

23. A method as claimed in claim 22, wherein said vascularization compound is chosen from the group consisting of retinoic acid, Matrigel, laminin and laminin derived peptides.
